(19) United States
(12) Patent Application Publication
(10) Pub. No.: US 2013/0138134 A1
(43) Pub. Date: May 30, 2013

(54) STENT DEVICES FOR SUPPORT, CONTROLLED DRUG DELIVERY AND PAIN MANAGEMENT AFTER VAGINAL SURGERY

(71) Applicants: Noel M. Elman, Cambridge, MA (US); Costas A. Apostolis, Watertown, MA (US)

(72) Inventors: Noel M. Elman, Cambridge, MA (US); Costas A. Apostolis, Watertown, MA (US)

(21) Appl. No.: 13/697,349

(22) PCT Filed: May 10, 2011
(86) PCT No.: PCT/IB2011/052045

§ 371 Date: Dec. 12, 2012

Related U.S. Application Data
(60) Provisional application No. 61/334,176, filed on May 13, 2010.

(51) Int. Cl. A61M 29/02

(52) U.S. Cl. CPC A61M 29/02
USPC 606/193

(57) ABSTRACT

Stent devices comprising an inflatable inner balloon defined by an envelope and an outer balloon surrounding the inner balloon concentrically, the outer balloon serving as a therapeutic agent reservoir used for controlled therapeutic agent delivery. In some embodiments, the outer balloon has two separate walls which define its volume and is independently inflatable. In some embodiments, the two concentric balloons form a substantially cylindrical structure which has a proximal end and a distal end. A catheter carrying inflation and drainage lumens extends outward from the proximal end. In some embodiments, the distal end includes an embedded cervix accommodating tip or has a non-embedded cervix accommodating tip attached thereto. The stent devices can be used to maintain the integrity and placement of vaginally placed mesh or graft after reconstructive procedures, prevent vaginal hematoma formation and bleeding, provide pain control after surgery and deliver antibiotics or hormones to the vaginal epithelium.

### CROSS REFERENCE TO RELATED APPLICATIONS

This application claims priority from U.S. Provisional Patent Application No. 61/334,176 filed May 13, 2010 and titled “Device to provide mechanical stability post vaginal surgery, and provide controlled drug delivery, including pain management”, which is incorporated herein by reference in its entirety.

### FIELD AND BACKGROUND

Embodiments of the invention relate in general to post surgery medical devices and more particularly to inflatable devices that provide support, controlled drug delivery and pain management after vaginal surgery.

Gynecological surgery is one of most common types of surgery performed in the USA. Female pelvic medicine and reconstructive surgery is a fairly new field, developed for the treatment of female pelvic disorders which include pelvic organ prolapse, urinary incontinence, chronic and acute pelvic pain, defecatory dysfunction and neurologic disorders of the genitourinary system. In the past five years, pelvic reconstructive surgery has taken a dramatic turn to involving the use of biological grafts and polypropylene mesh (also referred to herein as “reconstruction implants”) in reconstructive procedures. This has significantly increased post surgery success rates and has improved the quality of life of millions of women worldwide. Polypropylene mesh is an inert material which helps reconstruct and suspend the apical, anterior and posterior vaginal compartments in women with pelvic organ prolapse. It has become an excellent option for use in pelvic reconstruction when appropriate.

Some of the complications faced by pelvic reconstructive surgeons when using biological grafts or polypropylene mesh for vaginal reconstruction include the maintenance of appropriate placement of the mesh or graft for in-growth of native tissue to take place, bleeding and hematoma formation, mesh erosions, and post-operative pain management. Success rates after reconstructive procedures vary according to the nature of patient tissue and the type of the reconstruction implant material. Failure to adequately support reconstruction implants can lead to folding and excessive contraction of the implanted graft or mesh, and to pelvic pain and disruption of the repair. After an incision is closed by means of sutures, there is a critical time window in which applied pressure is required to improve surgical success.

In present practice, a heavy sterile gauze used as vaginal packing serves as the mainstay of post surgical prevention of the abovementioned potential complications. The use of gauze is painful and the gauze is abrasive and not particularly effective in preventing post vaginal surgery complications. There are a number of known inflatable stent-devices with single and/or double inflatable balloons suggested for use in various post surgery procedures in body cavities similar to the vagina. Among these are devices disclosed in U.S. Pat. Nos. 6,520,977, 7,708,716 and 7,220,252 and in U.S. Patent Application 2008/0215031. However, these known stent devices do not provide post vaginal surgery support after reconstructive procedures using grafts or mesh, do not provide adjustable compression to achieve hemostasis, do not provide uniform, controlled drug delivery, and do not provide controlled quantification of blood loss or cervicovaginal secretions. Also, known stent devices often require suturing for positioning.

There is therefore an existing, unmet clinical need for mechanical support of the anterior vaginal wall following pelvic reconstructive surgery utilizing polypropylene mesh or biological tissue repairs. It would be advantageous to have stent devices which can provide post vaginal surgery support, specifically after reconstructive procedures using grafts or mesh, as well as assistance in postoperative recovery, including pain management. It would further be advantageous to have localized and/or controlled drug delivery of anesthetics, antibiotics, hemostatic agents and tissue growth promoting factors, which can provide a clinical advantage for a more successful recovery after vaginal surgery.

### SUMMARY

In various embodiments, there are provided stent devices which comprise an inflatable inner balloon defined by an envelope. The inner balloon is surrounded concentrically by an outer balloon. In some embodiments, the outer balloon serves as a therapeutic agent (drug) reservoir for controlled drug delivery. Hereinafter, “therapeutic agent” and “drug” are used interchangeably. In some embodiments, the two concentric balloons form a substantially cylindrical structure. The cylindrical structure is characterized by a proximal end and a distal end. Each device includes a catheter carrying inflation/deflation and drug supply lumens which are in fluid communication with the two balloons. Optionally, the catheter may also carry a drainage lumen and separate cervix accommodating tip (see below) inflation/deflation lumens.

In some embodiments, the outer balloon has two (internal and external) walls, wherein the internal wall is separate from the inner balloon envelope. This structure is particularly advantageous in that is provides a defined volume for the drug reservoir independent of the inner balloon volume, and ensures more uniform distribution of the drug(s) in the reservoir. It also ensures hermetic sealing of the drug reservoir. In some embodiments, at least part of the external wall is a porous membrane used to release one or more drugs in a controlled fashion. In some embodiments, at least a non-porous part of the external wall is coated, impregnated, or otherwise covered with a therapeutic agent, which is releasable to the surrounding tissue. The controlled drug delivery may be used for post-operative pain management, hormone administration or local antibiotic administration. In some embodiments, the external wall may include an anti-slip pattern to prevent expulsion of the device (which may exemplarily result from vaginal lubrication).

A device according to an embodiment of the invention may be used to maintain the integrity and placement of vaginally placed mesh or graft after reconstructive procedures; prevent vaginal hematoma formation and bleeding; provide pain control after surgery, and/or deliver antibiotics or hormones to the vaginal epithelium. In some embodiments, such a device may adopt several shapes to obtain a very close anatomical fit to the inner walls of the vagina as well as to the cervix. In some embodiments, a device disclosed herein may further include at a distal end a cervix accommodating tip (also referred to simply as “tip”). The tip may be “embedded”, i.e. integrated with the inner balloon, or “non-embedded”, i.e. as a separate component attached to the distal end. A non-embedded tip may be inflatable or non-inflatable.

When inflatable, it may include one or two (internal and external in a radial direction) sections. When including two sections, an external section may be adapted for controlled drug delivery. The cervix accommodating tip is particularly advantageous in that it allows for a more secure placement of the device around the cervix, as well as for better support of the apex of the vagina. The device can thus be secured within the vagina without the need for sutures. In some embodiments which include a cervix accommodating tip, the inner balloon envelope may serve as the inner wall of the drug reservoir.

When inflated or filled, a device according to an embodiment of the invention adapts to the shape of the vagina walls to provide stability to a mesh or graft and to enable controlled drug delivery.

### BRIEF DESCRIPTION OF DRAWINGS

Aspects, embodiments and features of the invention will become apparent from the following detailed description when considered in conjunction with the accompanying drawings.

In the drawings:

FIG. 1A shows an external view of a two-balloon, three-wall device according to an embodiment of the invention device in a collapsed state;

FIG. 1B shows a longitudinal cross section view of the device in FIG. 1A in a collapsed state;

FIG. 1C shows an external view of the device in FIG. 1A in an inflated state;

FIG. 1D shows a longitudinal cross section view of the device in FIG. 1A in an inflated state;

FIG. 1E shows the device in FIG. 1A in an isomeric view;

FIG. 1F shows the device in FIG. 1C in an isomeric view;

FIG. 2 shows a schematic longitudinal cross section view of a two-balloon, two-wall device according to another embodiment of the invention, the device having an embedded non-inflatable cervix accommodating tip;

FIG. 3A shows an external view of a two-balloon, three-wall device with an embedded non-inflatable cervix accommodating tip in a collapsed state, according to an embodiment of the invention;

FIG. 3B shows a longitudinal cross section view of the device in FIG. 3A in the same collapsed state;

FIG. 3C shows an external view of the device in FIG. 3A in an inflated state;

FIG. 3D shows a longitudinal cross section view of the device in FIG. 3A in an inflated state;

FIG. 3E shows the device in FIG. 3A in an isomeric view;

FIG. 3F shows the device in FIG. 3C in an isomeric view;

FIG. 4A shows a schematic longitudinal cross section view of a sleeve-less, two-balloon, two-wall device according to another embodiment of the invention, the device having an embedded inflatable cervix accommodating tip;

FIG. 4B shows a schematic longitudinal cross section view of a device similar to the one in FIG. 4A, but including a sleeve;

FIG. 5 shows a longitudinal cross section view of a two-balloon, three-wall device according to yet another embodiment of the invention, the device having an embedded inflatable cervix accommodating tip;

FIG. 6 shows a longitudinal cross section view of a two-balloon, two-wall device according to yet another embodiment of the invention, the device having an external, non-embedded and non-inflatable cervix accommodating tip;

FIG. 7 shows a cross sectional view of a two-balloon, three-wall device according to an embodiment of the invention, the device having an external, non-embedded and non-inflatable cervix accommodating tip;

FIG. 8 shows a longitudinal cross section view of a two-balloon, two-wall device according to yet another embodiment of the invention, the device having an external, inflatable, one-section cervix accommodating tip;

FIG. 9 shows a longitudinal cross section view of a two-balloon, two-wall device according to yet another embodiment of the invention, the device having an external, inflatable, two-section cervix accommodating tip;

FIG. 10 shows a longitudinal cross section view of a two-balloon, three-wall device according to yet another embodiment of the invention, the device having an external, inflatable, two-section cervix accommodating tip;

FIG. 11 shows a device according to an embodiment of the invention inserted into the vaginal cavity and accommodating the cervix.

### DETAILED DESCRIPTION

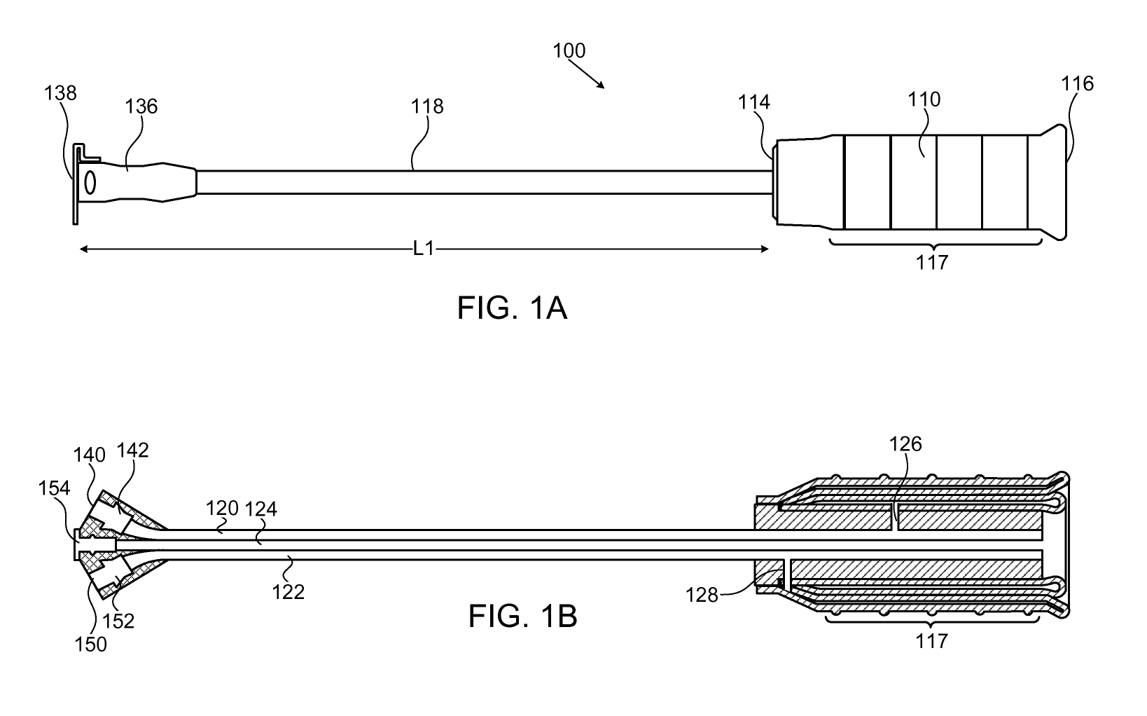

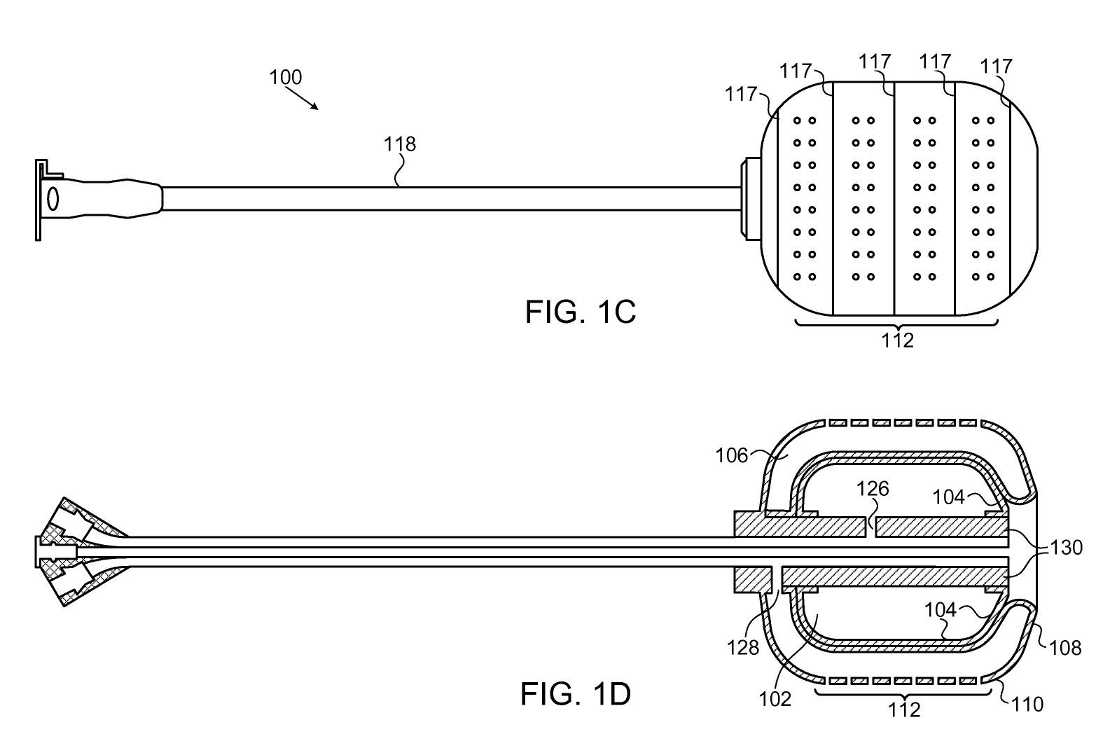

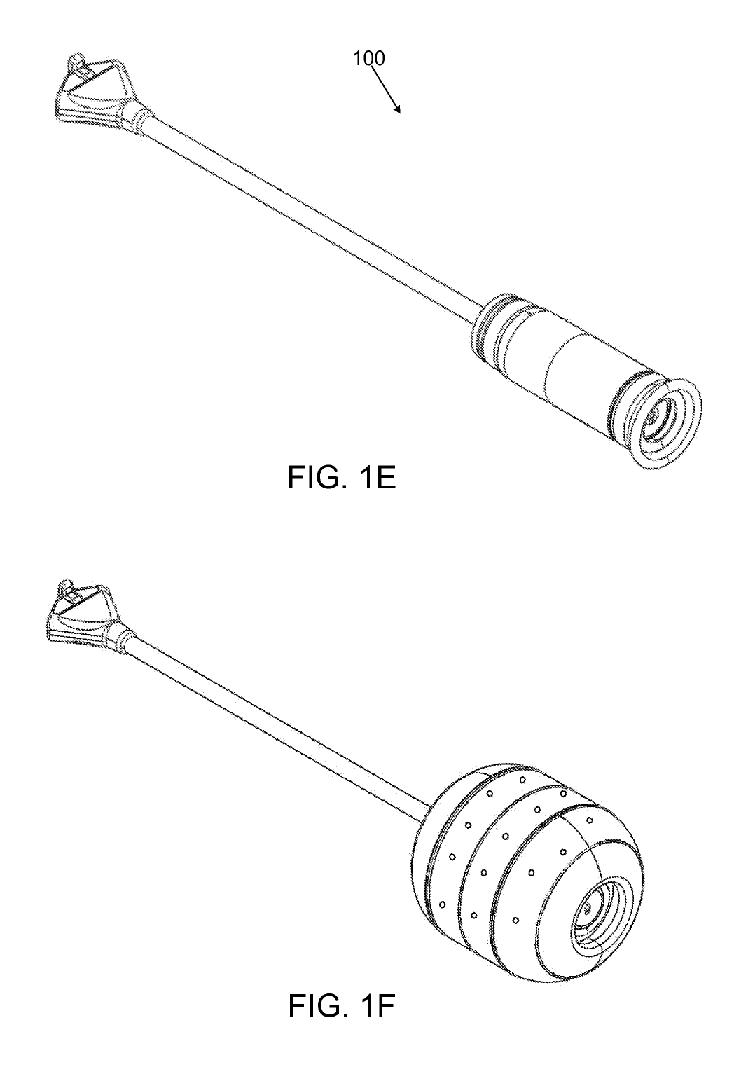

FIGS. 1A-1F show, in various views, a device 100 according to one embodiment of the invention. FIG. 1A shows an external view of device 100 in a collapsed state. FIG. 1B shows a longitudinal cross section view of device 100 in the same collapsed state. FIG. 1C shows an external view of device 100 in an inflated state. FIG. 1D shows a longitudinal cross section view of device 100 in the same inflated state. FIGS. 1E and 1F show isomeric views of, respectively, the devices in FIGS. 1A and 1C.

Device 100 includes an inner balloon 102 with an envelope 104, surrounded by an outer balloon 106 which has an internal wall 108 and an external wall 110. This structure is referred to henceforth as a “two-balloon, three-wall” structure. In some embodiments, the inner balloon serves as an inflation or “compression” balloon, while the outer balloon serves as a drug reservoir and is inflatable and capable of controlled drug delivery to surrounding tissue. Outer balloon 106 is hermetically sealed from inner balloon 102. In an embodiment, outer balloon 106 is generally shaped to follow the shape of inner balloon 102. In the embodiment shown in FIGS. 1, in both collapsed and inflated states, inner balloon 102 and outer balloon 106 form a substantially cylindrical and concentric structure which has a proximal end 114 and a distal end 116. In an inflated state, outer balloon 106 may exemplarily have a length of about 6-7 cm and a diameter of about 6-8 cm. Other sizes outside these ranges are of course possible to fit a particular anatomy.

In some embodiments, at least part of the external wall includes a porous membrane 112. Membrane 112 is perforated with a pattern of predetermined shape and size perforations (holes) used to release one or more drugs from the drug reservoir (106) to surrounding tissue in a controlled fashion (“controlled drug release”). Balloons 102 and 106 may be made of any biocompatible and flexible material, for example any elastomic or elastoplastic polymeric material such as silicone, rubber, urethane, latex, ethylene vinyl acetate, polyisoprene, polyamide elastomer, polyester elastomer, styrenic elastomer, polytetrafluoro elastomer, polyvynil chloride, etc., as well as mixtures thereof. In some embodiments, the two balloons may be made of the same material. In other embodiments, the two balloons may be made of different materials. In some embodiments, external wall 110 may optionally include anti-slip features or patterns 117 which allow adherence to the vaginal epithelium and prevent slippage or expulsion of the device from the vaginal canal. The anti-slip features may include protrusions, ribs, bumps, folds, shallow channels, or other two- or three-dimensional structures formed on the external balloon wall facing body tissue.

Device 100 further includes a catheter 118 which may extend part of, or the entire length of the cylindrical structure between proximal end 114 and distal end 116. The catheter also extends out of proximal end 114 to a desired length L1. Exemplarily, L1 may be about 10-14 cm. The catheter has a diameter large enough to accommodate various lumens and provides structural support (mechanical stiffness) to the inner balloon. In device 100, catheter 118 includes three lumens: an inner balloon inflation/deflation lumen 120 for inflating and holding the inner balloon to a required volume and for subsequently deflating it; an outer balloon inflation/deflation lumen 122 which may be used for independently inflating, deflating and/or inserting one or more drugs into outer balloon 106; and an optional drainage/irrigation lumen 124 used to provide drainage of any cervical secretions and bleeding. Each balloon is in fluid communication with its respective inflation/deflation lumen through vias, such as vias 126 for inner balloon 102 and vias 128 for outer balloon 106. The inner balloon may be inflated using a medium such as air, gas, liquid, gel or polymer. The outer balloon may be filled with a drug-carrying aqueous solution or gel, which can diffuse through the porous membrane to deliver the drug to the required tissue.

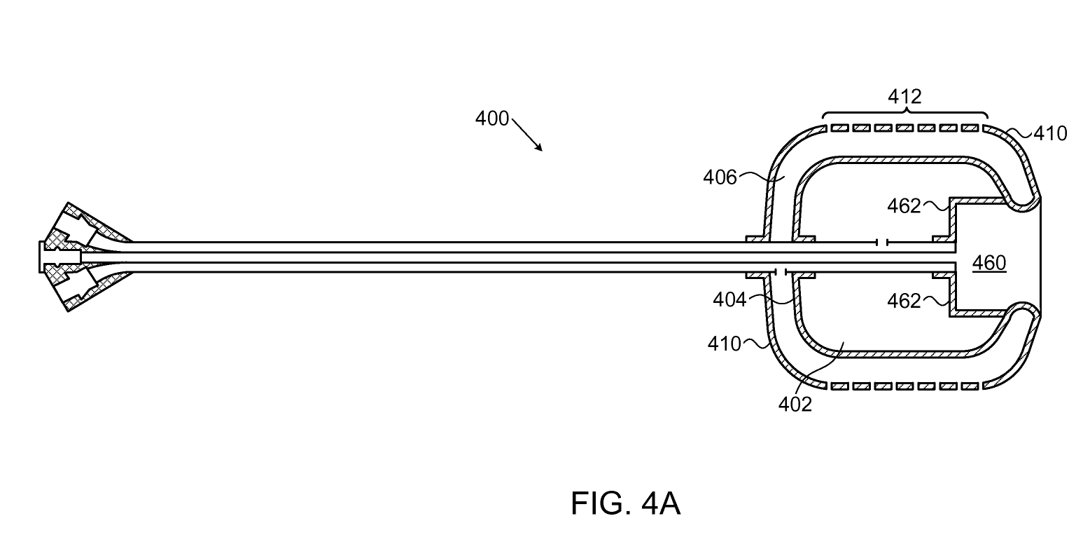

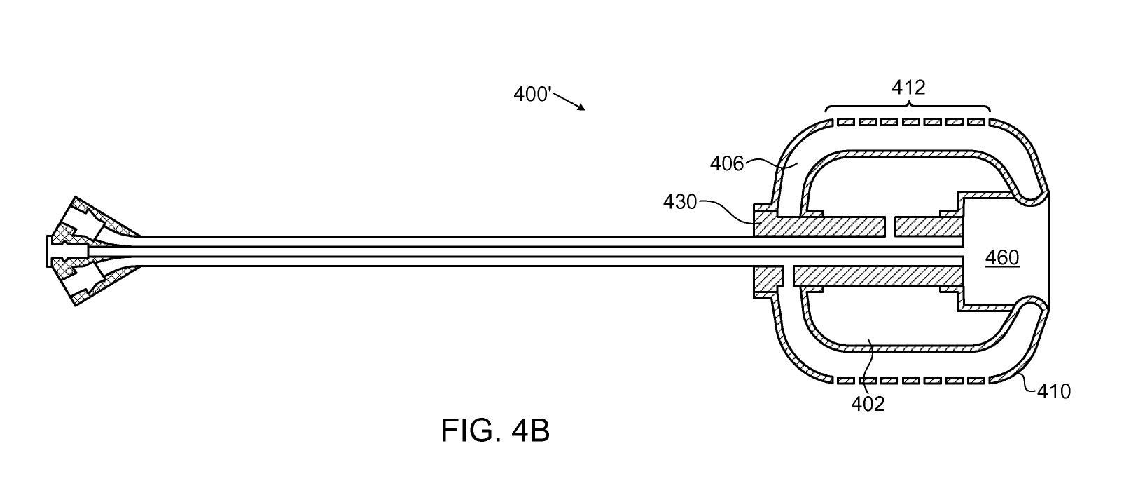

Device 100 may optionally further include a sleeve 130 as a structural element concentrically defined around the catheter inside the balloon structure. The sleeve may be made of a flexible material, such as an elastomer. In the embodiment shown, the two balloons are bonded to the sleeve. A sleeve is advantageous in that it provides a flexible support and a radial offset from the catheter for the balloons to be inflated. The radial offset may exemplarily be in the 1-3 cm range. This structure is still slim enough for ease of insertion into, and retrieval from the vaginal cavity. The radial offset allows the balloons to be inflated from a greater radial support. Typical balloons made of elastometers (e.g. silicone) become shape distorted, e.g. a desired cylindrical shape becomes a pear shape when large volumes (e.g. greater than 10 cc) are required for inflation. The offset provided by a sleeve allows less inflation volume for the balloons to prevent such a shape distortion. Note however that in some embodiments without a sleeve (for example in FIG. 4A), the balloons may be bonded directly to the catheter. The vias connect the lumens to the volumes to be inflated/deflated through the catheter wall and/or the sleeve, as necessary.

The inflation/deflation lumens may be coupled to external ports at a proximal end 136 of catheter 118 through pressure valves. As shown in FIGS. 1, lumen 120 is coupled to an inner balloon port 140 through a valve 142, while lumen 122 is coupled to an outer balloon port 150 through a valve 152. Lumen 124 is hooked directly to a drainage port 154. Optionally, end 136 is covered by a cap 138 which provides control over the drainage and maintains fluids within the device, if required. When the cap is removed, the drainage port can be attached to an external drainage bag (not shown) for collection of fluids. A Luer lock adapter tip (not shown) may be used to connect the ports to a standard type syringe to facilitate inflation or deflation of both balloons.

Focusing now on the structure, function and advantages of outer balloon 106, this balloon can be filled with one or more therapeutic agents. Exemplary therapeutic agents include hemostatic and vaso-constricting agents, anesthetic solutions, anti-inflammatory agents, tissue growth factors, chemotherapeutic agents, and antibiotics. The therapeutic agents may be stored in the drug reservoir in solid-phase (lyophilized), as a liquid, or as a gel. The double-wall structure of outer balloon 106 (walls 108, 110) provides a number of significant advantages over known stent devices:
a) The two walls define a volume which is independent of the (inflated) volume of inner balloon 102. That is, the outer balloon volume is decoupled from the inner balloon volume. The inner balloon volume is pressure-defined by the medical practitioner, who inflates it to fit anatomical differences among patients. The decoupling of the outer balloon from the (variable) inner balloon volume allows the outer balloon to be filled to a pre-defined drug content.
b) The drug can be uniformly distributed within the outer balloon volume without depending on the inner balloon to provide the uniformity by means of compression.
c) The elastic properties and structure of the balloon materials can be independently tailored for particular extensions. Exemplarily, the outer balloon can be pre-formed to a desired non-compressed volume. This feature can provide a better approximation of the desired volume without the need for additional expansion. Further exemplarily, the outer balloon can be defined to have a large diameter in its relaxed state, whereas the inner balloon can provide the complementary compression to obtain a conformal anatomical adaptation. The thickness of internal wall 108 is independent of, and can be different from that of inner balloon envelope 104. While the expansion of the inner balloon depends on the thickness of its envelope (a thinner envelope is more flexible and allows more expansion), the expansion of the outer balloon depends on the thicknesses of its two walls.
d) The permeability of a membrane is proportional to porosity and inversely proportional to its thickness. Therefore, the diffusion of the therapeutic agents across membrane 112 is controllable through adjustment of its thickness, which equals that of external wall 110. The therapeutic agent diffusion is also expected to be more stable, since the drug reservoir volume is more uniformly distributed, preventing air bubbles from blocking some pores.
e) The double-wall structure is further advantageous in that the drug reservoir is more reliable and safe, since even if the inner balloon explodes, internal wall 108 renders the drug reservoir less susceptible to structural damage. In summary, the advantageous features of the novel double-wall structure of the external drug delivery balloon disclosed herein are numerous and significant.

In use after vaginal reconstructive surgery or for treating an atrophic condition of the vagina, the device is inserted into the vaginal cavity without penetrating into the cervix. The two balloons are inflated to assume the shape of the vaginal cavity, and to apply pressure against the walls of the vagina. The inflation sequence may start with either balloon. As mentioned, the inner balloon is used to apply pressure on the vaginal walls as a stabilizing measure, such as for example after vaginal mesh reconstruction, to control hemorrhage, secretion, or discharge. The outer balloon may be used to provide further pressure as well as a controlled state of consistent drug delivery to the vaginal epithelium.

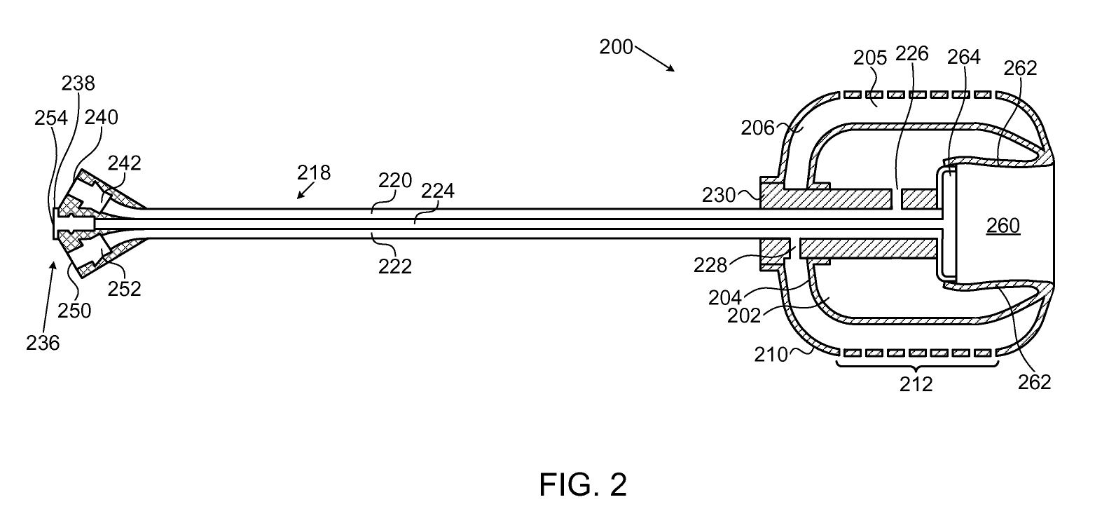

FIG. 2 shows a longitudinal cross section view of a device 200 according to another embodiment of the invention. Device 200 includes an inner balloon 202 with an envelope 204 and an outer balloon 206 which has envelope 204 as its internal wall, in contrast with outer balloon 106 of FIGS. 1, which has two separate walls. Thus, the drug reservoir in device 200 is defined by the space 205 between envelope 204 and an external wall 210. This structure is referred to henceforth as a “two-balloon, two-wall” structure. At least part of external wall 210 includes a porous membrane 212. The inner and outer balloons serve the same purposes as in device 100. Each balloon is in fluid communication with its respective inflation/deflation lumen through vias such as vias 226 for inner balloon 202 and vias 228 for outer balloon 206. Optionally, device 200 includes a sleeve 230 similar to sleeve 130. Device 200 further includes a catheter 218, which includes three lumens 220, 222 and 224, which are similar to lumens 120, 122 and 124 of device 100 in structure and function. As in device 100, the inflation/deflation lumens may be hooked to respective ports 240 and 250 through respective valves 242 and 252, while the drainage lumen may be hooked directly to a drainage port 254, with a proximal end 236 of catheter 218 optionally covered by a cap 238. As in device 100, external wall 210 may optionally include anti-slip features or patterns (not shown).

In further contrast with device 100, device 200 includes an embedded cervix accommodating tip 260. In this embodiment, tip 260 has a flexible (non-compressible) wall 262.

As used herein, “embedded” means that tip 260 is an integral part of inner balloon 202, i.e. it resides within the balloon structure. Optionally, the cervical accommodating tip may be structurally coupled to the inner catheter structure by means of a flexure 264. The flexure may be a thinner, more flexible section of inner balloon envelope 204 attached to the catheter or sleeve, may be defined with the same mold as the thicker inner balloon envelope, and may be made of the same material, e.g. silicone. Alternatively, the flexure can be a separate diaphragm or disk attached at an inner diameter perimeter to the catheter or sleeve and at an external perimeter surface to the inner balloon envelope. The flexure assists in fitting the device to accommodate the cervix and in removal of the device after use, thereby providing enhanced operability. Tip 260 may have a size and shape designed to anatomically fit a variety of cervices. The tip may be used for a number of purposes in different procedures: to help support the vaginal sidewalls and apex of the vagina after pelvic reconstructive surgery using reconstructive implants; after routine vaginal surgery for added hemostatic control and controlled drug delivery to vaginal epithelium; to accommodate the cervix in order to allow for detection of drainage of any bleeding or discharge after surgical repair; for post-cervical biopsies to replace the current standard of gauze packing or Monsel solution; or to provide compression to the cervix in the event of a cervical laceration following obstetrical vaginal delivery, which has been surgically repaired and considered hemostatic by the operating surgeon.

The cervix accommodating tip provides an exceptional and novel approach to vaginal mesh or graft repairs in that it completely accommodates the cervix in order to allow the device to extend beyond the cervix to support the apex of the vagina. This innovative feature allows for compression of the vaginal tissue against the mesh or graft beyond the cervix, thereby maintaining the reconstructive repair in an anatomical position for in-growth of native tissue to take place. Devices including such tips are unique in their capability to help maintain adequate vaginal apical support after vaginal reconstructive surgery.

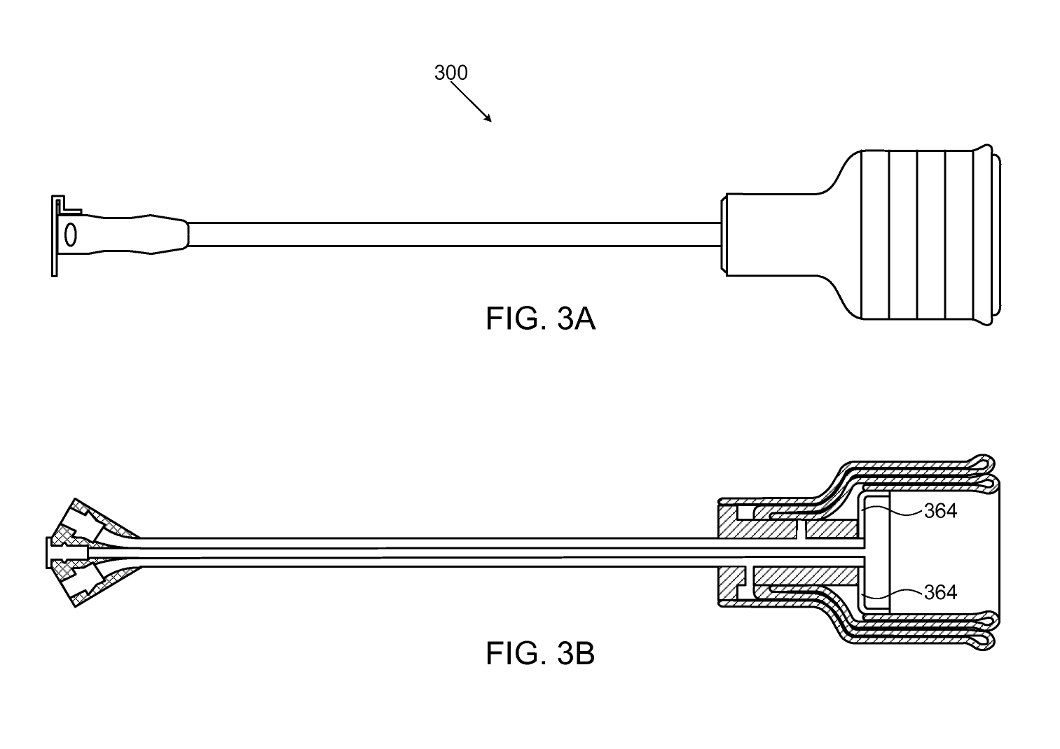

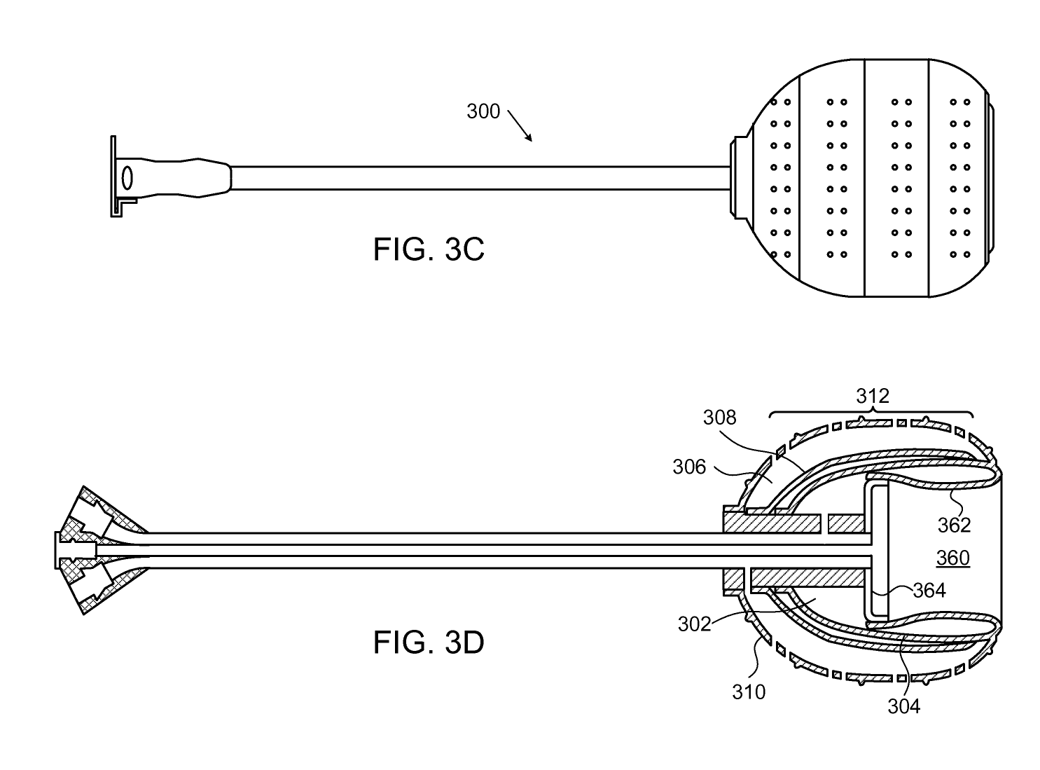

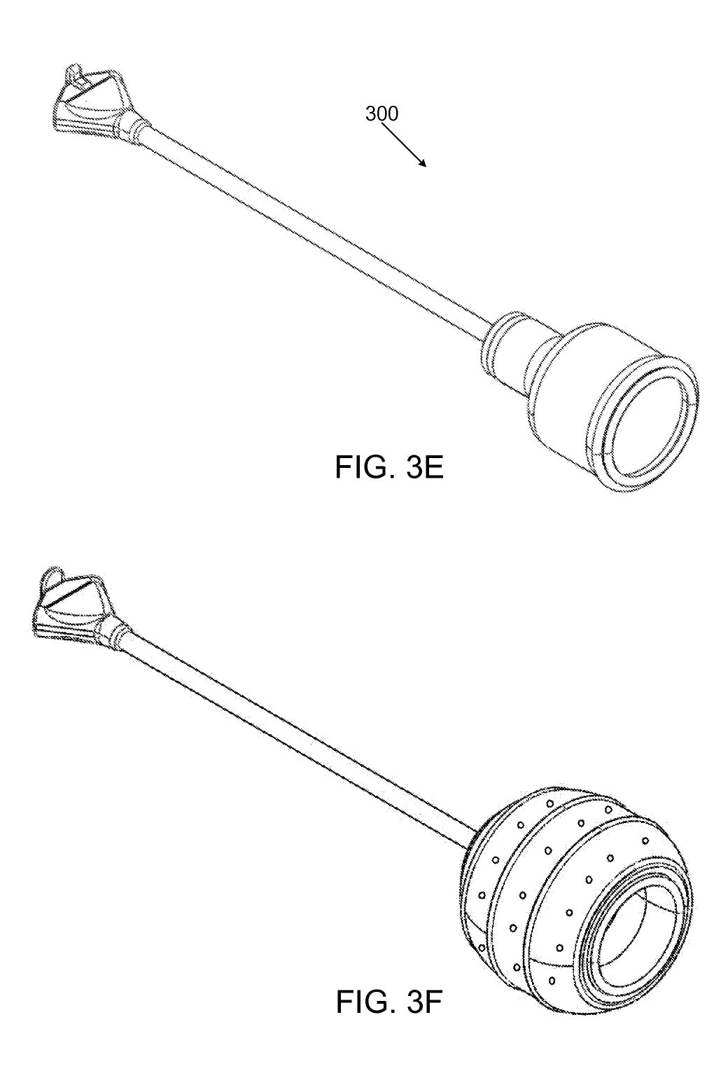

FIGS. 3A-3F show in various views, a device 300 according to yet another embodiment of the invention. FIG. 3A shows an external view of device 300 in a collapsed state. FIG. 3B shows a longitudinal cross section view of device 300 in the same collapsed state. FIG. 3C shows an external view of device 300 in an inflated state. FIG. 3D shows a longitudinal cross section view of device 300 in the same inflated state. FIGS. 3E and 3F show isomeric views of, respectively, the devices in FIGS. 3A and 3C.

Similar to device 100, device 300 includes a “three-wall” structure of an inner balloon 302 with an envelope 304, surrounded by an outer balloon 306 with an internal wall 308 and an external wall 310, at least part of which external wall includes a porous membrane 312. The inner and outer balloons serve the same purposes as in device 100. Devices 100 and 300 have in common a number of components, including the catheter, lumens, optional sleeve, valves, ports, vias, cover cap and optional anti-slip features, which are therefore not numbered and not described in detail. However, in contrast with device 100 and similar to device 200, device 300 further includes an embedded, cervix accommodating tip 360 with a non-compressible wall 362 and a flexure 364. Tip 360 may have a size and shape designed to anatomically fit a variety of cervices and can be used for a number of purposes in different procedures, as described for device 200 above.

FIGS. 4A and 4B show a longitudinal cross section view of a device according to yet another embodiment of the invention. FIG. 4A shows a device 400 which does not include a sleeve, while FIG. 4B shows a device 400 which includes a sleeve 430. Devices 400 and 400′ are similar to device 200 in that they have a two-balloon (402 and 406), two-wall (404 and 410) structure and include an embedded cervix accommodating tip 460. At least part of external wall 410 includes a perforated membrane 412. Devices 200, 400 and 400′ have in common a number of components, including the catheter, lumens, valves, ports, vias, cover cap and optional anti-slip features, which are therefore not numbered and not described in detail. In device 400, the balloons are bonded directly to the catheter, while in device 400′ they are bonded to the sleeve. In contrast with non-inflatable tip 260 in device 200, tip 460 has an inflatable section 462 which is a continuation of inner balloon 402. Thus, pressure applied to inner balloon 402 acts on wall 462 to compress wall 462 around the cervix to provide a more secure fit. The collapsed state of devices 400 and 400′ is similar to the collapsed state of device 100.

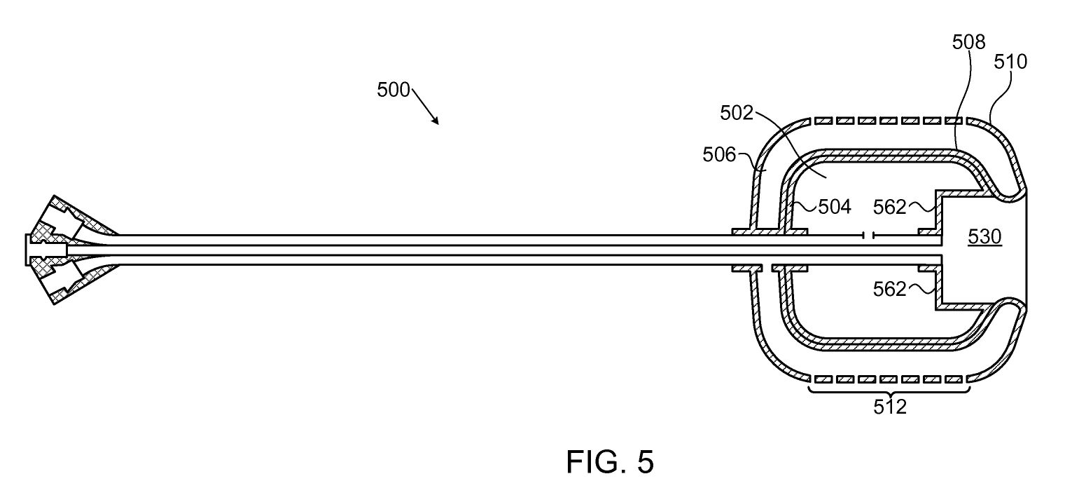

FIG. 5 shows a longitudinal cross section view of a device 500 according to yet another embodiment of the invention. Device 500 is similar to device 300 in that it has a two-balloon (502 and 506), three-wall (504, 508 and 510) structure and includes an embedded cervix accommodating tip 560. At least part of external wall 510 includes a perforated membrane 512. Devices 300 and 500 have in common a number of components, including the catheter, lumens, optional sleeve, valves, ports, vias, cover cap and optional anti-slip features, which are therefore not numbered and not described in detail. However, in contrast with device 300 and similar to device 400, tip 560 has an inflatable section 562 which is a continuation of inner balloon 502. Thus, pressure applied to inner balloon 502 acts on wall 562 to compress wall 562 around a cervix inserted in the tip to provide a more secure fit. The collapsed state of device 500 is similar to the collapsed state of device 300.

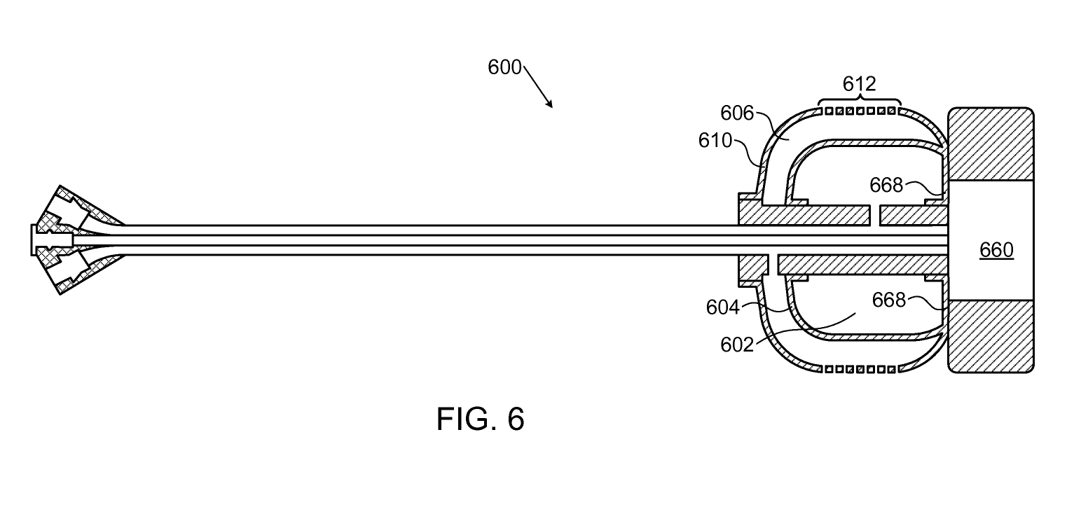

In some embodiments, the cervix accommodating tip may be a “non-embedded” unit, not integrated in the balloon structure. FIG. 6 shows a longitudinal cross section view of a device 600 according to yet another embodiment of the invention. Device 600 is similar to device 200 or 400 in that it has a two-balloon (602 and 606), two-wall (604 and 610) structure. At least part of external wall 610 includes a perforated membrane 612. However, in contrast with devices 200 and 400, device 600 includes a non-embedded, non-inflatable cervix accommodating tip 660. Tip 660 may be a solid ring, made of a material similar to that of the balloons, e.g. silicone or any other material mentioned above. The ring has an appropriate, anatomically fitting shape to accommodate the cervix. Tip 660 is attached to a section 668 of external wall 610 at the distal end of the two-balloon structure by appropriate means (for example by gluing or thermal bonding). Alternatively, the tip may be attached or to a section of the catheter or sleeve (not shown). Device 600 further includes a number of components common with previously described devices (e.g. 200 or 400) including the catheter, lumens, optional sleeve, valves, ports, vias, cover cap and optional anti-slip features, which are therefore not numbered and not described in detail.

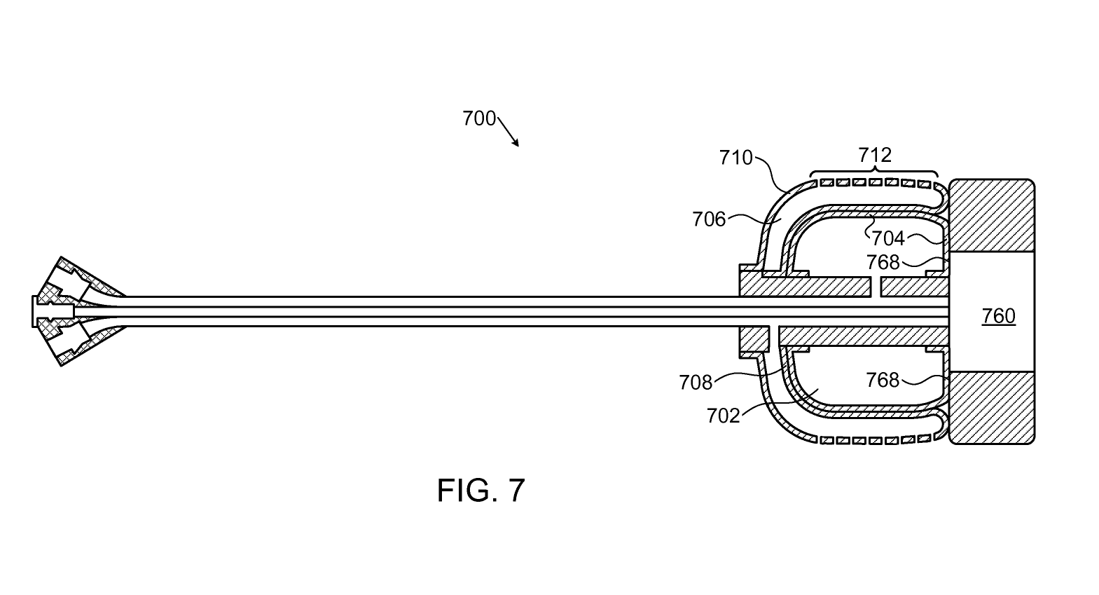

FIG. 7 shows a longitudinal cross section view of a device 700 according to yet another embodiment of the invention. Device 700 is similar to device 300 or 500 in that it has a two-balloon (702 and 706), three-wall (704, 708 and 710) structure. However, in contrast with devices 300 and 500, device 700 includes a non-embedded, non-inflatable cervix accommodating tip 760 similar to tip 660 of device 600. As in device 600, tip 760 is attached to a section 768 of external wall 710 at the distal end of the two-balloon structure by appropriate means. Alternatively, the tip may be attached or to a section of the catheter or sleeve (not shown). Device 700 further includes a number of components common with previously described devices (e.g. 300 or 500) including the catheter, lumens, optional sleeve, valves, ports, vias, cover cap and optional anti-slip features, which are therefore not numbered and not described in detail.

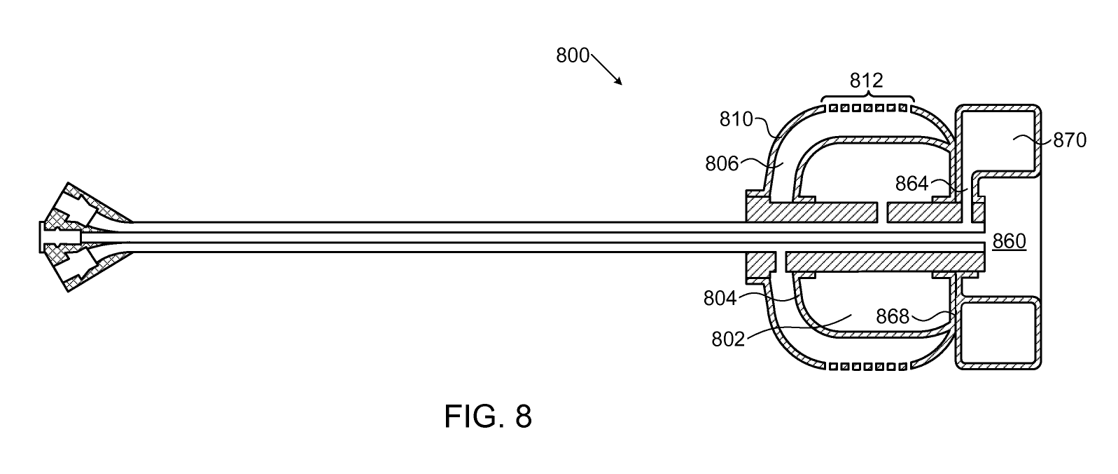

FIG. 8 shows a longitudinal cross section view of a device 800 according to yet another embodiment of the invention. Device 800 is similar to device 600 in that it has a two-balloon (802 and 806), two-wall (804 and 810) structure. At least part of external wall 810 includes a perforated membrane 812. However, in contrast with device 600, device 800 includes an inflatable cervix accommodating tip 860 with an inflatable ring or cylinder shaped section 870. Section 870 is inflatable through vias 864 which may communicate with the inner balloon inflation/deflation lumen. Alternatively, section 870 may be inflatable via a separate inflation/deflation lumen (not shown). The independent inflation/deflation capability of the tip provides increased flexibility in adjusting (and providing compression) to the cervix and in supporting compression of the vaginal wall. Tip 860 is attached to a section 868 of external wall 810 at the distal end of the two-balloon structure by appropriate means. Alternatively, the tip may be attached or to a section of the catheter or sleeve (not shown). Device 800 further includes a number of components common with previously described devices (e.g. 600) including the catheter, lumens, optional sleeve, valves, ports, vias, cover cap and optional anti-slip features, which are therefore not numbered and not described in detail.

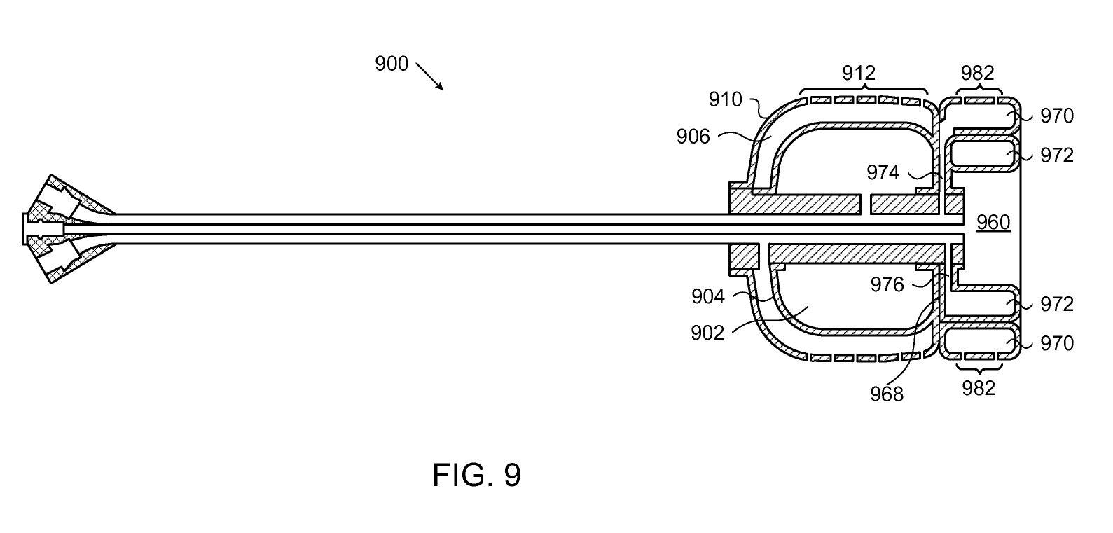

FIG. 9 shows a longitudinal cross section view of a device 900 according to yet another embodiment of the invention. Device 900 is similar to device 800 in that it has a two-balloon (902 and 906), two-wall (904 and 910) structure with a non-embedded and inflatable cervix accommodating tip 960. At least part of external wall 910 includes a perforated membrane 912. However, in contrast with tip 860 in device 800, tip 960 includes a separate drug reservoir 970 surrounding an inner inflatable section 972 and having an external wall 980 at least partially in the form of a perforated membrane 982. Drug reservoir 970 and section 972 are inflatable through, respectively, vias 974 and 976. Alternatively, they can be inflated/deflated independently through separate lumens (not shown). Independent inflation/deflation of the two tip sections provides increased flexibility in drug delivery, in adjusting (and providing compression) to the cervix and in supporting compression of the vaginal wall. Tip 960 is attached to a section 968 of external wall 910 at the distal end of the two-balloon structure by appropriate means. Alternatively, the tip may be attached or to a section of the catheter or sleeve (not shown). Device 900 further includes a number of components common with previously described devices including the catheter, lumens, optional sleeve, valves, ports, vias, cover cap and optional anti-slip features, which are therefore not numbered and not described in detail.

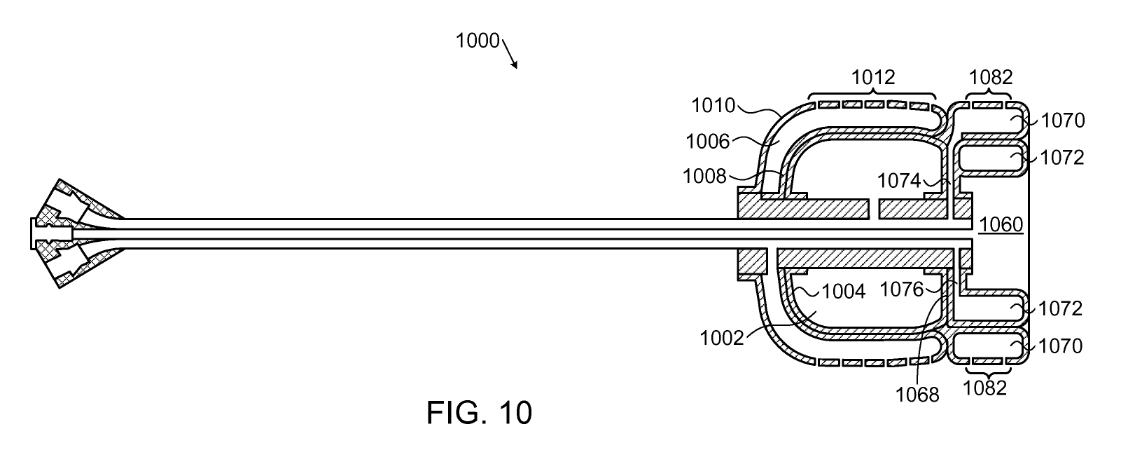

FIG. 10 shows a longitudinal cross section view of a device 1000 according to yet another embodiment of the invention. Device 1000 is similar to device 300 or 500 in that it has a two-balloon (1002 and 1006), three-wall (1004, 1008 and 1010) structure and to device 900 in that it has a non-embedded, two-section, inflatable cervix accommodating tip 1060. In common with tip 960, tip 1060 includes a separate drug reservoir 1070 surrounding an inner inflatable section 1072 and having an external wall 1080 at least partially in the form of a perforated membrane 1082. Drug reservoir 1070 and section 1072 are inflatable through, respectively, vias 1074 and 1076. Alternatively, they can be inflated/deflated independently through separate lumens (not shown). As shown, drug reservoir section 1070 has two separate walls 1084 and 1086. In another embodiment (not shown) the drug reservoir section of the tip may lack wall 1084. Device 1000 further includes a number of components common with previously described devices including the catheter, lumens, optional sleeve, valves, ports, vias, cover cap and optional anti-slip features, which are therefore not numbered and not described in detail.

The device embodiments in FIGS. 1-10 have been described with reference to an external balloon (drug reservoir) from which a drug can be controllably dispensed to surrounding tissue through a perforated membrane. In additional embodiments, any of the devices in FIGS. 1-10 can have an external surface coated, impregnated of otherwise covered with a therapeutic agent, instead of, or in addition to the perforated membrane. Thus, solid sections of external walls 110, 210, 310, 410, 510, 610, 710, 810, 910 and 1010 may be impregnated with a therapeutic agent (not shown). Devices according to such embodiments, while compromising on the controllable aspect of drug delivery, provide still significant advantages over known devices in terms of flexibility and efficiency of operation.

### Examples of Clinical Use

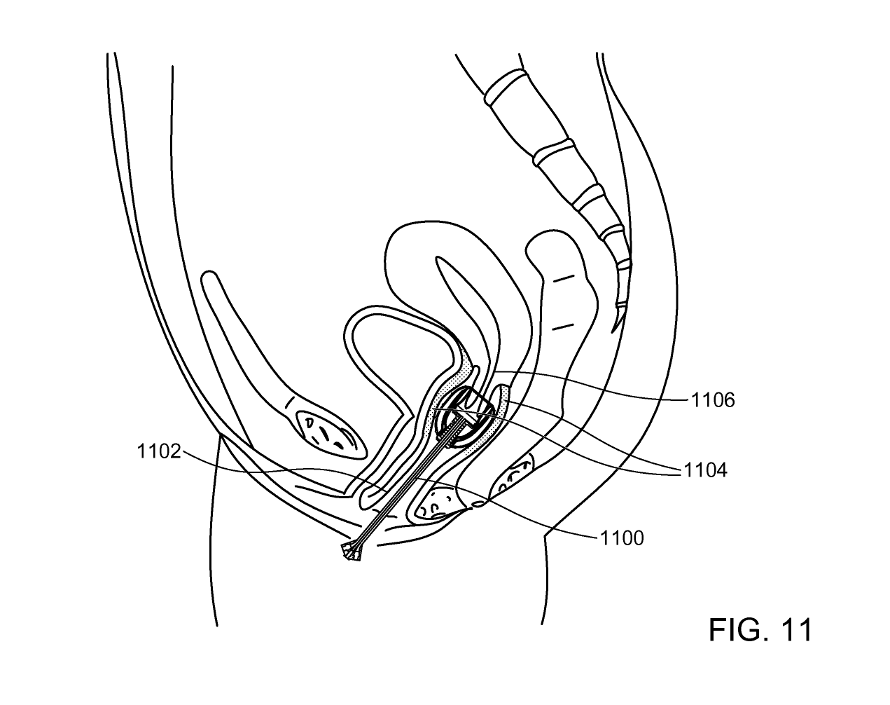

A device disclosed herein may be simply operated by positioning the device in its collapsed state, in a desired position in a body cavity such as a vaginal cavity, and inflating the inner balloon to a desired pressure and volume. FIG. 11 shows a device 1100 according to an embodiment of the invention in an inflated state and inserted into a vaginal cavity 1102. Device 1100 presses against a mesh 1104 and accommodates a cervix 1106. Optimum pressure is obtained as determined by the operating surgeon so as to secure the device against the vaginal epithelium. Care should be taken not to overinflate so as to compromise blood flow to the vaginal epithelium. Drug delivery is effected by pressurizing the inner balloon. Various physical mechanisms contribute to the drug release from the reservoir, including diffusion, osmotic pressure and differential pressure. The inflation of the outer balloon (when present) affects the drug delivery as well since the internal pressure of the reservoir can be greater than the outside pressure. This provides a pressure gradient to force drugs through the pores. In pelvic surgery, after an appropriate length of time (days), the inner balloon can be deflated and the device removed.

In obstetrical uses, a device disclosed herein may be used to apply compression to the vaginal epithelium, post vaginal delivery, in the instance of (but not limited to) a lateral sulcus or other vaginal laceration including cervical lacerations. The compression can control potential hematoma formation and excessive blood loss. In general, the use will occur after surgical repair and control of bleeding. In gynecological uses, a device disclosed herein may be used to apply compression to the vaginal epithelium after certain repairs or biopsies performed in an operating room or office setting which include (but are not limited to) vaginal reconstruction, cervical biopsies, or vaginal tears. A device may be further used to provide compression and control of potential hematoma formation and excessive blood loss. A device can be also used to aid in the release of medication in patients with conditions such as atrophic vaginitis requiring hormone administration, for vaginal infections such as yeast or bacterial infections requiring antibiotic administration, or anesthetic medication for pain control after outpatient surgical procedures.

In urogynecology/pelvic reconstructive surgery, a device disclosed herein may be used to apply compression to the vaginal epithelium after certain repairs or biopsies performed in an operating room or office setting. These repairs or biopsies may include (but are not limited to) vaginal reconstructive surgery requiring synthetic mesh repairs or biological graft augmentation. Compression may be also applied to control potential hematoma formation and excessive blood loss to prevent disruption of the repair. The device can be further used to provide anesthetic medication for pain control after above mentioned procedures.

In gynecology, a device disclosed herein may be used for antibiotic or hormone administration in instances of vaginal infections such as bacterial vaginosis, vaginal candidiasis, or atrophic vaginitis. The device could be placed into the vagina and filled with a certain antibiotic or hormone preparation.

In laparoscopic surgery, a device disclosed herein may be used in the vagina to help maintain pneumoperitoneum during total laparoscopic hysterectomy. Upon removing the uterus and cervix from the vaginal epithelium, the device could be placed into the vagina and inflated to help occlude the vaginal defect and loss of the CO₂ pneumoperitoneum, thereby facilitating closure of the vaginal cuff.

### Exemplary Drug Delivery Treatment

A device as described in any of the embodiments above can be used for stabilization of the vaginal epithelium after vaginal reconstructive surgery and for pain control as follows: Typically, the desired therapeutic duration of the lidocaine (“t_{target}”) is approximately 24 hours after surgery. In order to obtain a therapeutic level, the size and number of perforations (orifices) in the porous membrane is chosen according to target therapeutic values. Exemplarily, a standard 30 cc lidocaine gel can be used, and diffusion can be used as an approximation of the mass transport mechanism from the reservoir to the targeted area outside of the device if both external and internal pressures are equal. The flux J from a single orifice in the drug reservoir is given by:

J=−D(ΔC/Δx) (1)

where D is the diffusivity constant (for lidoicaine from gel, ca. 10e⁻¹⁰ m²/s), ΔC is the change in concentration (approximately 2×10⁴ g/m³ for commercial lidocaine available at 20 mg/mL) and Δx is the thickness of the membrane (exemplarily a 1 mm-thick silicone layer). The release rate R from a single orifice is given by:

R=J×A (2)

where A is the circular orifice area=π×r² (where for example r=2 mm). The diffusion time t to empty the reservoir is given by:

t=G/R=G/(J×A) (3)

where G is the mass of the drug (in grams). The number of orifices can be defined as:

n=t/t_{target} (4)

For the defined example, J=2×10⁻³ g/s−m²; R=6×10⁻⁸ g/s; t=2 G/6×10⁻⁸=3×10⁷ s and n=3×10².

All patents and patent applications mentioned in this specification are herein incorporated in their entirety by reference into the specification, to the same extent as if each individual patent or patent application was specifically and individually indicated to be incorporated herein by reference. In addition, citation or identification of any reference in this application shall not be construed as an admission that such reference is available as prior art.

While the invention has been described with respect to a limited number of embodiments, it will be appreciated that many variations, modifications and other applications of the invention may be made. For example, stent devices disclosed herein may be modified for use in rhinoplasty surgery or in colorectal surgery. For example, in alternative embodiments of a device of the invention, the roles of the inner and outer balloons may be reversed, with the inner balloon serving as a drug reservoir and the outer balloon serving for compression. In such embodiments, appropriate fluid communication channels (not shown) are provided between the inner balloon volume and the external wall, to facilitate controlled drug delivery. Such modifications and variations are covered by the above teachings and are within the purview of the appended claims without departing from the spirit and intended scope of the invention.

1-45. (canceled)

46. A stent device for providing support after vaginal surgery comprising:
a. an inflatable inner balloon having an envelope;
b. an inflatable outer balloon surrounding the inner balloon, the outer balloon having an internal wall in contact with the inner balloon envelope and an external wall adapted to release a non-ablative therapeutic agent in a controlled way to surrounding body tissue, wherein at least one of the inner or outer balloons is enabled to apply compression to the surrounding body tissue; and
c. a catheter including a first lumen in fluid communication with the inner balloon and a second lumen in fluid communication with the outer balloon, the first lumen adapted to provide a first inflation medium to the inner balloon and the second lumen adapted to provide the non-ablative therapeutic agent to the outer balloon.

47. The stent device of claim 46, wherein the inner and outer balloons form a substantially concentric structure having a proximal end and a distal end.

48. The stent device of claim 47, wherein the catheter further includes a third lumen used to provide drainage of body secretions and of bleeding.

49. The stent device of claim 46, further comprising a sleeve concentrically surrounding the catheter, the sleeve providing a flexible support and a radial offset for the two balloons.

50. The stent device of claim 46, further comprising anti-slip features on the external wall.

51. A stent device for providing support after vaginal surgery comprising:
a. an inflatable inner balloon having an envelope;
b. an inflatable outer balloon surrounding the inner balloon, the inner and outer balloons forming a substantially concentric structure having a proximal end and a distal end, wherein at least one of the inner or outer balloons is enabled to apply compression to surrounding body tissue;
c. a catheter including a first lumen in fluid communication with the inner balloon and a second lumen in fluid communication with the outer balloon, the first lumen adapted to provide a first inflation medium to the inner balloon and the second lumen adapted to provide a therapeutic agent medium into the outer balloon; and
d. a non-occluding cervix accommodating tip positioned at the distal end, the non-occluding cervix accommodating tip having a size and shape adapted to accommodate a cervix.

52. The stent device of claim 51, wherein the outer balloon includes an external wall adapted to release the therapeutic agent in a controlled way to the surrounding body tissue.

53. The stent device of claim 52, wherein the outer balloon further includes an internal wall in contact with the inner balloon envelope, thereby providing a closed space of defined outer balloon volume which is unaffected by an inflated inner balloon volume.

54. The stent device of claim 51, wherein at least an external surface of the cervix accommodating tip is adapted to release therapeutic agent in a controlled way to the surrounding body tissue.

55. The stent device of claim 51, wherein the cervix accommodating tip is embedded and is further adapted to apply compression to the cervix.

56. The stent device of claim 51, wherein the cervix accommodating tip is a separate non-embedded structure attached to the distal end.

57. The stent device of claim 56, wherein the cervix accommodating tip includes a single solid section.

58. The stent device of claim 56, wherein the cervix accommodating tip includes an inner inflatable section and an outer inflatable section and is further adapted to apply compression to the cervix.

59. The stent device of claim 51, further comprising anti-slip features on the external wall.

60. The stent device of claim 51, further comprising a sleeve concentrically surrounding the catheter, the sleeve providing a flexible support and a radial offset for the two balloons.

61. A method for providing support after vaginal surgery, comprising the steps of:
a. inserting a stent device into the vaginal cavity of a patient, the vaginal cavity surrounded by vaginal epithelium, the stent device comprising:
i. an inflatable inner balloon having an envelope,
ii. an inflatable outer balloon surrounding the inner balloon, the outer balloon having an internal wall in contact with the envelope and an external wall adapted to release a non-ablative therapeutic agent in a controlled way to the vaginal epithelium, and
iii. a catheter including a first lumen in fluid communication with the inner balloon and a second lumen in fluid communication with the outer balloon, the first lumen adapted to provide a first inflation medium to the inner balloon and the second lumen adapted to provide the non-ablative therapeutic agent to the outer balloon; and
b. using the stent device to apply compression to the vaginal epithelium to prevent a post-surgical complication resulting from the vaginal surgery.

62. The method of claim 61, wherein the inner and outer balloons form a substantially concentric structure having a proximal end and a distal end and wherein the stent device further comprises a non-occluding cervix accommodating tip positioned at the distal end, the non-occluding cervix accommodating tip having a size and shape adapted to accommodate a cervix and optionally to apply compression to the cervix.

63. The method of claim 61, wherein the vaginal surgery includes use of a biological or polypropylene mesh or graft, and wherein the step of using the stent device includes maintaining an appropriate placement of the mesh or graft to allow in-growth of native tissue.

64. The method of claim 61, wherein the step of using the stent device includes inflating at least one of the inner or outer balloons to apply the compression to the vaginal epithelium and to release the non-ablative therapeutic agent in a controlled way to the vaginal epithelium through the external wall.

65. The method of claim 61, wherein the post vaginal surgery includes a repair surgical procedure selected from the group consisting of repair of vaginal sulcus, repair of vaginal lacerations and repair of cervical lacerations.
